(19) United States
(12) Patent Application Publication
(10) Pub. No.: US 2005/0070711 A1
(43) Pub. Date: Mar. 31, 2005

(54) Method for reducing residual alcohols in crystalline valacyclovir hydrochloride

(76) Inventors: Igor Lifshitz, Petach-Tiqva (IL); Ben-Zion Dolitzky, Petach Tiqva (IL)

Correspondence Address: KENYON & KENYON, ONE BROADWAY, NEW YORK, NY, 10004, US

(21) Appl. No.: 10/688,538

(22) Filed: Oct. 16, 2003

Related U.S. Application Data
(60) Provisional application No. 60/419,270, filed on Oct. 16, 2002.
(60) Provisional application No. 60/427,320, filed on Nov. 18, 2002.

(51) Int. Cl. C07D 473/10

(52) U.S. Cl. 544/276

(57) ABSTRACT

Provided is a method of reducing excess residual process solvents, especially excess residual process alcohol (e.g. isopropanol), in valacyclovir hydrochloride to about 1000 ppm (weight) or less by contacting valacyclovir hydrochloride having excess residual process alcohol with moist gas.

### CROSS-REFERENCE TO RELATED APPLICATIONS

This application claims the benefit of U.S. Provisional Application Ser. No. 60/419,270, filed Oct. 16, 2002 and U.S. Provisional Application Ser. No. 60/427,320, filed Nov. 18, 2002, both of which are incorporated herein by reference.

### BACKGROUND OF THE INVENTION

Valacyclovir is an L-valyl ester prodrug of acyclovir. Acyclovir is an acyclic analog of a natural nucleoside which has been found to have high anti-viral activity. Acyclovir is widely used in the treatment and prophylaxis of viral infections in humans, particularly infections caused by the herpes group of viruses. See Goodman and Gilman's, The Pharmacological Basis of Therapeutics 1193-1198 (9th ed. 1996).

Acyclovir is an acyclic guanine nucleoside analog that lacks a 3′-hydroxyl on the side chain. Acyclovir has the chemical name 6H-Purin-6-one, 2-amino-1,9-dihydro-9-[(2-hydroxyethoxy)methyl]. (CAS Registry No.59277-89-3.) Acyclovir as the sodium salt is currently marketed as ZOVIRAX®. The chemical structure of acyclovir is shown as Formula I.

Valacyclovir has the chemical name 1-valine, 2-[(2-amino-1,6-dihydro-6-oxo-9H-purin-9-yl)methoxy]ethyl ester. (CAS Registry No. 124832-26-4.) Valacyclovir is currently marketed as VALTREX®. The chemical structure of valacyclovir is shown as Formula I.

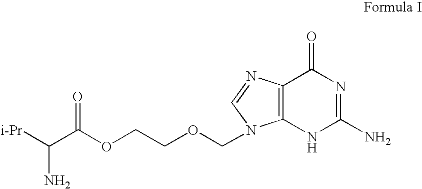

For oral administration, it is advantageous to administer valacyclovir rather than acyclovir because acyclovir is poorly absorbed from the gastrointestinal tract after oral administration in both animals and humans. In contrast, valacyclovir is rapidly absorbed from the gastrointestinal tract after oral administration. Moreover, valacyclovir is converted rapidly and virtually completely to acyclovir after oral administration in healthy adults. The conversion of valacyclovir is thought to result from first-pass intestinal and hepatic metabolism through enzymatic hydrolysis.

Work-up, isolation, and purification procedures for valacyclovir hydrochloride can and frequently do use solvents that are or that contain alcohols such as methanol, ethanol or iso-propanol. U.S. Pat. No. 4,957,924 discloses one such crystallization procedure that uses ethanol. In such cases, when alcohols are used in work-up or other procedures, the valacyclovir hydrochloride can contain 5000 ppm or more of excess residual process alcohol. The presence of unnecessary foreign substances, for example excess residual process alcohols, in any active pharmaceutical ingredient (API) is undesirable. These excess residual process alcohols are not necessary to the efficacy of the API valacyclovir hydrochloride. The solvents may be toxic and can produce undesirable effects in the patient receiving valacyclovir hydrochloride. Since there is no therapeutic benefit from residual process: solvents, all residual solvents should be removed to the extent possible to meet quality-based requirements.

Indeed, health regulatory agencies in many countries have established limits for foreign substances in active pharmaceutical ingredients and may require manufacturers to adapt manufacturing procedures to reduce or eliminate them. For example, the United States Food and Drug Administration has promulgated guidelines (Q3C) that apply to residual solvents in drug substances and drug products.

The International Conference on Harmonization of Technical Requirements for Registration of Pharmaceuticals for Human Use has also promulgated Draft Guidelines (Q3C) for Residual Solvents in Pharmaceuticals. See Step 4 Draft, Jul. 16, 1997, Dr. Shigeo Kojima, rapporteur (hereafter ICH Guidelines). The draft proposes three classes of solvents and several options for quantifying the permissible level of them. Class 3 solvents should be limited (Option 1) to 5000 ppm, provided that the total daily dosage would be less than 50 mg (concentration in tablet should not exceed 5000 ppm). Ethanol and the propanols are among the class 3 solvents that should be limited by good manufacturing procedures (GMP).

Although some residual process solvent in an API or drug product may be an unavoidable consequence of the manufacturing process, the level of residual process solvent should be reduced to a minimum. Clearly, methods for reducing excess process solvents like alcohols in valacyclovir hydrochloride to a level less than 5000 ppm are needed.

### SUMMARY OF THE INVENTION

In one aspect, the present invention relates to a method of reducing excess residual process solvent (>5000 ppm), especially excess residual process alcohol, in valacyclovir hydrochloride having excess residual process solvent, including the step of statically or dynamocally contacting the valacyclovir hydrochloride having excess residual process alcohol with a humid gas, especially humid air.

In another aspect, the present invention relates to a method of reducing excess residual process alcohol, especially excess residual process isopropanol, in valacyclovir hydrochloride having excess residual process alcohol including the step of dynamically contacting such valacyclovir hydrochloride, with humid air, especially humid air of≧50% relative humidity, more especially≧75% relative humidity, in a fluiduzed bed apparatus.

### DETAILED DESCRIPTION OF THE INVENTION

One skilled in the art of the synthesis of organic compounds understands that solvents, e.g. alcohols, are often used in synthesis procedures and that traces, sometimes substantial traces, of these solvents can remain in the compound synthesized. The remaining solvents, which can be referred to as residual process solvents, can be difficult to remove.

Residual process solvents in pharmaceutical compounds (and ultimately any pharmaceutical compositions prepared therefrom) serve no therapeutic purpose and can be harmful to the patient. As discussed above, governmental regulatory agencies and international advisory organizations have promulgated regulations and guidelines for residual (process) solvents in pharmaceutical compounds. Excess residual process solvent in valacyclovir hydrochloride is defined in relation to the concentration limits set for class 3 solvents, of which iso-propanol is on example, by the ICH Guidelines. Accordingly, excess process alcohol in valacyclovir hydrochloride refers to process alcohol, especially ethanol and iso-propanol, in excess of 5000 ppm on a weight basis. Valacylcovir hydrochloride having excess residual process alcohol refers to valacyclovir hydrochloride having 5000 ppm or more, on a weight basis, residual process alcohol. Valacyclovir hydrochloride having excess residual process alcohol is the preferred starting material for use in the practice of the method of the present invention.

Alcohols can be used as solvents in the synthesis, work-up, and purification of valacyclovir hydrochloride. The present invention provides a method for reducing the excess residual process alcohol content of crystalline valacyclovir hydrochloride having excess residual process alcohols, for example ethanol, n-propanol, or iso-propanol, remaining from, for example, work-up, isolation, or other treatment procedures, for example recrystallization. Valacyclovir hydrochloride is considered to have excess residual process alcohol if the residual process alcohol is≧5000 ppm on a weight basis. The present method includes the step of contacting (exposing) particles (e.g. crystals) of valacyclovir hydrochloride having excess residual process alcohol with a humid gas, preferably at ambient atmospheric pressure (about 750 to about 765 mm Hg).

The valacyclovir hydrochloride having excess residual process alcohol or process alcohol (residual process alcohol of 5000 ppm or more) can be from any source. Typically, the valacyclovir hydrochloride will be obtained from a process in which an alcohol or alcohol-containing solvent is used, for example from a crystallization procedure in which an alcohol is used. But valacyclovir hydrochloride having excess residual process alcohol can be obtained directly from a synthesis process in which an alcohol is used. In such cases, the excess residual process alcohol can be more than 5000 ppm.

Any gas that does not induce or accelerate chemical degradation of valacyclovir hydrochloride during the treatment process can be used. Air is the preferred gas. Humid gas has a relative humidity (RH) of at least about 15%, preferably at least about 50%, more preferably at least about 75%. Relative humidity refers to the ratio (times 100) of the actual vapor pressure of water in a gas to the saturation vapor pressure of water in the gas at a particular temperature and pressure.

The contacting is conducted at ambient pressure and a temperature of about 10° C. to about 60° C. The contacting can be static or it can be dynamic.

In static contacting, particles (e.g. crystals) of valacyclovir hydrochloride are at rest. That is, they are not mechanically or otherwise agitated or stirred. Static contacting can be carried out, for example, by contacting particles of valacyclovir hydrochloride having excess residual process alcohol on a tray, preferably in a thin layer, with humid gas in a suitable enclosure such as, for example, a constant humidity chamber.

In dynamic contacting, particles of valacyclovir hydrochloride are in motion induced by mechanical or other agitation whilst being contacted with humid gas. Mechanical agitation can be provided by, for example, a ribbon-type blender through which humid gas is passed.

In a preferred embodiment, the valacyclovir hydrochloride having excess residual process alcohol is contacted with humid gas in a fluidized bed apparatus wherein the valacyclovir hydrochloride is fluidized with humid gas. Fluidized bed apparatus is well-known in the art. One example of suitable fluidized bed apparatus is a Retsch model TG-100

Valacyclovir hydrochloride having excess residual process alcohol is contacted with humid air for a contacting time sufficient to reduce the excess residual process alcohol to less that 5000 ppm, preferably to about 1000 ppm or less. The skilled artisan will know to optimize the contacting time by routine experimentation, taking into consideration factors such as the amount of excess residual process alcohol initially present, the size of the particles of valacyclovir hydrochloride, and the humidity of the humid gas. The higher the initial amount of excess residual process alcohol, the larger the particles of valacyclovir hydrochloride, and the lower the humidity of the humid gas, the longer will be, in general, the contacting time. The lower the initial amount of excess residual process alcohol and the higher the humidity of the humid gas, the shorter, in general, will be the contacting time.

By the method of the present invention, the excess residual process alcohol in valacyclovir hydrochloride is reduced to<5000 ppm, preferably to about 1000 ppm or less. Alcohol in valacyclovir hydrochloride can be measured by any means known in the art, for example by gas chromatography (GC).

The present invention can be illustrated with the following nonlimiting examples.

### EXAMPLE 1

Valacyclovir hydrochloride (about 10 g), crystallized from isopropanol/water and having about 6000 ppm excess residual process solvent, were dried in a fluidized bed drier—at about 40° C. for about 4 hours in a stream of humid air (ca. 80% RH). After drying, the material so treated contained less than about 300 ppm residual process solvent and about 9% by weight water.

1. A method for reducing excess residual process alcohol in valacyclovir hydrochloride having excess residual process alcohol comprising the step of contacting valacyclovir hydrochloride having excess residual process alcohol with a humid gas at ambient pressure.

2. The method of claim 1 wherein the humid gas is humid air.

3. The method of claim 2 wherein the humid air has a relative humidity of at least about 15%.

4. The method of claim 3 wherein the humid air has a relative humidity of at least about 50%.

5. The method of claim 4 wherein the humid air has a relative humidity of at least about 75%.

6. The method of claim 1 wherein the contacting is static.

7. The method of claim 1 wherein the contacting is dynamic.

8. The method of claim 7 wherein the contacting is in a fluidized bed apparatus.

9. The method of claim 1 wherein, after the exposing step, the residual process alcohol in the exposed valacyclovir hydrochloride is less than about 5000 ppm on a weight basis.

10. The method of claim 9 wherein the residual process alcohol in the exposed valacyclovir hydrochloride is about 1000 ppm or less on a weight basis.

11. The method of claim 1 wherein the process alcohol is ethanol.

12. The method of claim 1 wherein the process alcohol is iso-propanol.

13. A method for reducing excess residual process iso-propanol in valacyclovir hydrochloride having excess residual process iso-propanol comprising the step of contacting valacyclovir hydrochloride having excess residual process iso-propanol with humid air at ambient pressure in a fluidized bed apparatus, wherein the relative humidity of the air is at least about 15%.

14. The method of claim 13 wherein the relative humidity of the air is at least about 50%.

15. The method of claim 13 wherein the residual iso-propanol in the exposed valacyclovir hydrochloride is about 5000 ppm or less on a weight basis.

16. The method of claim 15 wherein the residual iso-propanol in the exposed valacyclovir hydrochloride is about 1000 ppm or less on a weight basis.

17. A method for reducing to about 1000 ppm on a weight basis or less the residual process alcohol in valacyclovir hydrochloride having greater than 1000 ppm on a weight basis residual process alcohol comprising the step of contacting, at ambient pressure, the valacyclovir hydrochloride having greater than 1000 ppm on a weight basis residual process alcohol with humid gas having a relative humidity of at least about 15%.

18. The method of claim 17 wherein the humid gas has a relative humidity of at least about 50%.

19. The method of claim 18 wherein the contacting is in a fluidized bed apparatus.
